(19) United States
(12) Patent Application Publication
(10) Pub. No.: US 2019/0239937 A1
(43) Pub. Date: Aug. 8, 2019

(54) Augmentation of Abscopal Effect of Cryotherapy and other Tumor Cell Death by Anti-angiogenic and Anti-tumor Vaccination

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 16/266,499

(22) Filed: Feb. 4, 2019

Related U.S. Application Data
(60) Provisional application No. 62/625,665, filed on Feb. 2, 2018.

(51) Int. Cl. A61B 18/02; A61P 35/00; A61K 39/00

(52) U.S. Cl. CPC A61B 18/02; A61P 35/00; A61K 45/06; A61K 39/001111; A61K 39/001131

(57) ABSTRACT

Disclosed are means, methods, and compositions, useful for augmenting immune response to tumor cell death occurring in the body, wherein the tumor cell death acts as a source of antigens to stimulate an anti-cancer immune response. In one embodiment of the invention cryosurgery is performed in a manner to facilitate immune mediated killing of distant tumors, in effect causing an abscopal reaction, the amplification of the abscopal reaction is performed by preimmunization with antigen compositions generated to target tumor endothelium. In another embodiment the invention teaches the amplification of abscopal effect by preimmunizing with placental and/or tumor antigens prior to induction of necrotic cell death through means such as cryoablation, hyperthermia, radiation therapy, radiation therapy and intravenous vitamin C, and chemotherapy.

### CROSS-REFERENCE TO RELATED APPLICATIONS

This Patent Application takes priority from Provisional Patent Application No. 62/625,665, titled Augmentation of Abscopal Effect of Cryotherapy and other Tumor Cell Death by Anti-angiogenic and Anti-Tumor Vaccination, filed on Feb. 2, 2018, the contents of which are expressly incorporated herein by this reference as though set forth in their entirety and to which priority is claimed.

### BACKGROUND OF THE INVENTION

In recent years there has been a growing realization that immune responses play a central role in cancer biology by eliminating many tumors at a very early stage and keeping those that avoid total elimination in a state of equilibrium, sometimes for many years. The eventual escape from this equilibrium phase with clinical manifestation of the disease is associated with dysregulated immune responses, manifesting, for example, as chronic inflammation or immune suppression. The strong and increasing evidence that the immune system is critically involved in the development, structural nature and progression of cancer has led to renewed interest in immunotherapeutic strategies for treatment of this class of diseases. To date, most attempts to develop such strategies have been based on the use of antigens derived from the patient's own tumor or from tumor cell lines and the transfer of ex-vivo expanded populations of tumor antigen-specific cytotoxic cells and antigen-presenting cells.

Cancer has been associated with inflammation since 1863, when Rudolf Virchow discovered leucocytes in neoplastic tissues and so made the first connection between inflammation and cancer. Since then, chronic inflammation has been deemed to be a risk factor for cancer. These reports demonstrate that an inflammatory environment supports tumor development and is consistent with that observed at tumor sites. However, the relationship of cancer with inflammation is not limited to the onset of the disease due to chronic inflammation. Schwartzberg proposed that chronic inflammation occurs due to tumor environment stress and that this generates a shield from the immune system. It has been recently demonstrated that the tumor microenvironment resembles an inflammation site, with significant support for tumor progression, through chemokines, cytokines, lymphocytes and macrophages which contribute to both the neovascularization and vasal dilation for increased blood flow, the immunosuppression associated with the malignant disease, and the establishment of tumor metastasis. Furthermore, this inflammation-site tumor-generated microenvironment, apart from its significant role in protection from the immune system and promotion of cancer progression, has an adverse effect on the success of current cancer treatments. Indeed, it has been found that the inflammatory response in cancer can compromise the pharmacodynamics of chemotherapeutic agents.

Moreover, metastatic cancer cells leave the tumor as microcolonies, containing lymphocytes and platelets as well as tumor cells Inflammation continues to play a role at metastatic sites by creating a cytokine milieu conducive to tumor growth. Immune homeostasis consists of a tightly regulated interplay of pro- and anti-inflammatory signals. For example, loss of the anti-inflammatory signals leads to chronic inflammation and proliferative signaling. Interestingly, cytokines that both promote and suppress proliferation of the tumor cells are produced at the tumor site. As in the case of cancer initiation, it is the imbalance between the effects of these various processes that results in tumor promotion.

It is believed that, to treat cancer, the most effective type of immune response is of a Type 1, which favors the induction of CD4+ Th1 cellular responses, and of CD8+ CTL responses. In the context of cancer vaccines, many immune stimulants are used, which promote the development of Th1 responses and are thought to inhibit the production of a Th2 response. To date, a major barrier to attempts to develop effective immunotherapy for cancer has been an inability to break immunosuppression at the cancer site and restore normal networks of immune reactivity. The physiological approach of immunotherapy is to normalize the immune reactivity so that the endogenous tumor antigens would be again recognized and effective cytolytic responses would be developed against cells bearing these antigens.

Anti-cancer immune responses accompanying the action of chemo- and radiotherapy have been recently reviewed and show that such responses are critical to therapeutic success by eliminating residual cancer cells and maintaining micro metastases in a state of dormancy. However, this reference makes it clear that there is no simple immunotherapeutic strategy available for consistently enhancing such immune responses.

There is evidence that therapeutic procedures that induce certain forms of immunogenic cancer cell death also lead to release of tumor antigens. There are three main types of cell death (Tesniere et al, Cell Death Differ 2008; 15:3-12): apoptosis (type 1), autophagy (type 2) and necrosis (type 3). Apoptosis, or programmed cell death, is a common and regular occurring phenomenon essential for tissue remodeling, especially in utero but also ex utero. It is characterized by DNA fragmentation in the nucleus and condensation of the cytoplasm to form ‘apoptotic bodies’ which are engulfed and digested by phagocytic cells. In autophagy, cell organelles and cytoplasm are sequestered in vacuoles which are extruded from the cell. Although this provides a means of survival for cells in adverse nutritional conditions or other stressful situations, excess autophagy results in cell death. Necrosis is a ‘cruder’ process characterized by damage to intracellular organelles and cell swelling, resulting in rupture of the cell membrane and release of intracellular material.

### DESCRIPTION OF THE INVENTION

The invention teaches means of activating abscopal effect in cancer patients and enhancing such systemic effects through immunizing against tumor endothelium. In some embodiments abscopal effect is induced by irradiation in a manner to cause localized tumor cell death. One of the first described examples of abscopal effect was published in 1975 when systemic melanoma metastasis started regressing after localized radiation treatment. Ohba et al reported the case of a 76-year-old Japanese man with hepatocellular carcinoma that regressed after radiotherapy for thoracic vertebral bone metastasis. Serum levels of tumor necrosis factor-alpha increased after radiotherapy. The findings suggests that such abscopal related regression may be associated with host immune response, involving cytokines such as tumor necrosis factor-alpha.

Another case was reported of a 69-year-old woman with advanced uterine cervical carcinoma with toruliform para-aortic lymph node metastases that showed an abscopal effect of radiation therapy (effect out of irradiated field). The patient was admitted to our University Hospital in March 2005, and treated with radiation therapy only for the primary pelvic lesions without chemotherapy because of her severe economic status. After the treatment, not only did the cervical tumor in the irradiated field disappear, but the toruliform para-aortic lymph node swelling outside the irradiated field also spontaneously disappeared. The patient is still alive and well without relapse.

Okuma et al reported on a 63-year-old Japanese man underwent extended right hepatic lobectomy for hepatocellular carcinoma. During his follow-up examination, a single lung metastasis and a single mediastinal lymph node metastasis were found. Trans-catheter arterial embolization was initially attempted to treat the mediastinal tumor, however this approach failed to take effect and carried risks of spinal artery embolism. External-beam irradiation, with a dose of 2.25 Gy per fraction, was performed using an antero-posterior parallel-opposed technique (total dose, 60.75 Gy). A computed tomography scan performed one month after starting radiotherapy showed a remarkable reduction of the mediastinal lymph node metastasis. In addition to this, they observed spontaneous shrinking of the lung metastasis, which was located in the right lower lobe and out of the radiation field. No chemotherapy was given during the period. There has been no recurrence of either the lung metastasis or the mediastinal lymph node metastasis during a follow-up 10 years after the radiotherapy. In another report an 80-year-old male with squamous cell carcinoma with bilobar hepatic metastases who underwent targeted Yttrium-90 radioembolization of the right hepatic lobe lesion. Subsequently, there was complete regression of the nontargeted, left hepatic lobe lesion.

Cases of abscopal effect have also been observed in chronic lymphocytic leukemia, Merkel Cell Carcinoma, melanoma, renal cell carcinoma, myeloma, pancreatic cancer, breast cancer, renal cell carcinoma, diffuse Giant tumor, and non-small cell lung cancer.

In a murine study, mice bearing a syngeneic mammary carcinoma, 67NR, in both flanks were treated with Flt3-L daily for 10 days after local radiation therapy (RT) to only 1 of the 2 tumors at a single dose of 2 or 6 Gy. The second nonirradiated tumor was used as indicator of the abscopal effect. Data were analyzed using repeated measures regression. Radiation therapy (RT) alone led to growth delay exclusively of the irradiated 67NR tumor, as expected. Surprisingly, growth of the nonirradiated tumor was also impaired by the combination of RT and Flt3-L. As control, Flt3-L had no effect without RT. Importantly, the abscopal effect was shown to be tumor specific, because growth of a nonirradiated A20 lymphoma in the same mice containing a treated 67NR tumor was not affected. Moreover, no growth delay of nonirradiated 67NR tumors was observed when T cell deficient (nude) mice were treated with RT plus Flt3-L. The authors concluded that the results of their experiments demonstrated that the abscopal effect is in part immune mediated and that T cells are required to mediate distant tumor inhibition induced by radiation.

Van der Meerans et al. reported on an investigation of the radiation-induced inflammatory response in C57BL6/J mice after total abdominal or total-body irradiation at a dose of 15 Gy. The goal was to determine the radiation-induced inflammatory response of the gut and to study the consequences of abdominal irradiation for the intestine and for the lungs as a distant organ. A comparison with total-body irradiation was used to take into account the hematopoietic response in the inflammatory process. For both irradiation regimens, systemic and intestinal responses were evaluated. A systemic inflammatory reaction was found after abdominal and total-body irradiation, concomitant with increased cytokine and chemokine production in the jejunum of irradiated mice. In the lungs, the radiation-induced changes in the production of cytokines and chemokines and in the expression of adhesion molecules after both abdominal and total-body irradiation indicate a possible abscopal effect of radiation in our model. The effects observed in the lungs after irradiation of the abdomino-pelvic region may be caused by circulating inflammatory mediators consequent to the gut inflammatory response.

The invention teaches augmentation of abscopal effect by immunization with proteins and/or peptides and/or other antigenic materials that induce immunity to the tumor endothelial cells. Other means of stimulating immunity to endogenous tumor antigens have been described in the art to augment abscopal effect. In one experiment tumors were implanted s.c. in the right or both flanks. After local irradiation at the right flank, ECI301, a human macrophage inflammatory protein-1alpha variant was injected i.v. Tumor volumes were measured every 3 days after treatment. In Colon26 adenocarcinoma-bearing BALB/c mice, repeated daily administration (over 3-5 consecutive days) of 2 mug per mouse ECI301 after local irradiation of 6 Gy prolonged survival without significant toxicity, and in about half of the treated mice, the tumor was completely eradicated. Three weekly administrations of ECI301 after local irradiation also led to significant, although less effective, antitumor radiation efficacy. ECI301 also inhibited growth of other syngeneic tumor grafts, including MethA fibrosarcoma (BALB/c) and Lewis lung carcinoma (C57BL/6). Importantly, tumor growth at the nonirradiated site was inhibited, indicating that ECI301 potentiated the abscopal effect of radiation. This abscopal effect observed in BALB/c and C57BL/6 mice was tumor-type independent. Leukocyte depletion studies suggest that CD8+ and CD4+ lymphocytes and NK1.1 cells were involved. Marked inhibition of tumor growth at the irradiated site, with complete tumor eradication and consistent induction of the abscopal effect, was potentiated by i.v. administration of ECI301. The results of this study may offer a new concept for cancer therapy, namely chemokine administration after local irradiation, leading to development of novel therapeutics for the treatment of advanced metastatic cancer. Another study aiming to augment the abscopal effect examined CTLA-4 blockade as a means of immune stimulation.

TSA mouse breast carcinoma cells were injected s.c. into syngeneic mice at two separate sites, defined as a “primary” site that was irradiated and a “secondary” site outside the radiotherapy field. When both tumors were palpable, mice were randomly assigned to eight groups receiving no radiotherapy or three distinct regimens of radiotherapy (20 Gy x 1, 8 Gy x 3, or 6 Gy x 5 fractions in consecutive days) in combination or not with 9H10 monoclonal antibody against CTLA-4. Mice were followed for tumor growth/regression. Similar experiments were conducted in the MCA38 mouse colon carcinoma model. In either of the two models tested, treatment with 9H10 alone had no detectable effect. Each of the radiotherapy regimens caused comparable growth delay of the primary tumors but had no effect on the secondary tumors outside the radiation field. Conversely, the combination of 9H10 and either fractionated radiotherapy regimens achieved enhanced tumor response at the primary site (P<0.0001). Moreover, an abscopal effect, defined as a significant growth inhibition of the tumor outside the field, occurred only in mice treated with the combination of 9H10 and fractionated radiotherapy (P<0.01). The frequency of CD8+ T cells showing tumor-specific IFN-gamma production was proportional to the inhibition of the secondary tumor. The authors concluded that fractionated but not single-dose radiotherapy induces an abscopal effect when in combination with anti-CTLA-4 antibody in two preclinical carcinoma models.

Similar results were observed when researchers attempted to improve the local and abscopal effect by modulating T cell immunity with systemic blockade of CTLA-4 signal. The growth of primary tumors was significantly inhibited by LRT while CTLA-4 antibody enhanced the antitumor effect. Growth delay of the second tumors was achieved when the primary tumor was radiated. LRT resulted in more T cell infiltration into both tumors, including Treg and cytotoxic T cells. Interestingly, the proportion of Treg over effector T cells in both tumors was reversed after CTLA-4 blockade, while CD8 T cells were further activated. The expression of the immune-related genes was upregulated and cytokine production was significantly increased. LRT resulted in an increase of TIT, while CTLA-4 blockade led to significant reduction of Tregs and increase of cytotoxic T cells in both tumors. The abscopal effect is enhanced by targeting the immune checkpoints through modulation of T cell immune response in murine mesothelioma.

Accordingly, in one embodiment of the invention addition of inhibitors to CTLA-4 such as antibodies, microbodies, siRNA, shRNA, antisense, shark or cameloid antibodies, or small molecules may be used together with tumor endothelial or tumor immunization means in order to augment abscopal effect of radiotherapy. Various concepts and regimens for utilization of CTLA-4 are known in the literature.

Use of another checkpoint inhibitor was reported to augment the abscopal effect. Researchers investigated the influence of PD-1 expression on the systemic antitumor response (abscopal effect) induced by stereotactic ablative radiotherapy (SABR) in preclinical melanoma and renal cell carcinoma models. We compared the SABR-induced antitumor response in PD-1-expressing wild-type (WT) and PD-1-deficient knockout (KO) mice and found that PD-1 expression compromises the survival of tumor-bearing mice treated with SABR. None of the PD-1 WT mice survived beyond 25 days, whereas 20% of the PD-1 KO mice survived beyond 40 days. Similarly, PD-1-blocking antibody in WT mice was able to recapitulate SABR-induced antitumor responses observed in PD-1 KO mice and led to increased survival. The combination of SABR plus PD-1 blockade induced near complete regression of the irradiated primary tumor (synergistic effect), as opposed to SABR alone or SABR plus control antibody. The combination of SABR plus PD-1 blockade therapy elicited a 66% reduction in size of nonirradiated, secondary tumors outside the SABR radiation field (abscopal effect). The observed abscopal effect was tumor specific and was not dependent on tumor histology or host genetic background. The CD11a(high) CD8(+) T-cell phenotype identifies a tumor-reactive population, which was associated in frequency and function with a SABR-induced antitumor immune response in PD-1 KO mice. It was concluded that SABR induces an abscopal tumor-specific immune response in both the irradiated and nonirradiated tumors, which is potentiated by PD-1 blockade.

While the classical immune checkpoints are known to be inhibitory cell surface molecules such as CTLA-4, PD-1, TIM-3, and LAG-3, cytokines can also inhibit abscopal effect. Accordingly, in one embodiment of the invention removal/neutralization of immune suppressive cytokines may also be performed. This is illustrated in one publication that may be used as a guide for one of skill in the art. In the publication researchers showed that antibody-mediated TGFβ neutralization during radiation therapy effectively generates CD8(+) T-cell responses to multiple endogenous tumor antigens in poorly immunogenic mouse carcinomas. Generated T cells were effective at causing regression of irradiated tumors and nonirradiated lung metastases or synchronous tumors (abscopal effect). Gene signatures associated with IFNγ and immune-mediated rejection were detected in tumors treated with radiation therapy and TGFβ blockade in combination but not as single agents. Upregulation of programmed death (PD) ligand-1 and -2 in neoplastic and myeloid cells and PD-1 on intratumoral T cells limited tumor rejection, resulting in rapid recurrence. Addition of anti-PD-1 antibodies extended survival achieved with radiation and TGFβ blockade. Thus, TGFβ is a fundamental regulator of radiation therapy's ability to generate an in-situ tumor vaccine. The combination of local radiation therapy with TGFβ neutralization offers a novel individualized strategy for vaccinating patients against their tumors.

In one aspect of the invention, augmentation of immunity to cancer may be accomplished by addition of passive antibodies to cancer cells prior to, concomitant with, or subsequent to radiation therapy. The use of passive antibodies is described in the literature and these protocols may be utilized together with endothelial cell vaccination, such as vaccination with ValloVax, in order to augment therapeutic effects. For example, combined treatment comprised of local irradiation and anti-neu antibody of tumor-bearing BALB/c mice significantly improved mouse survival (P<0.5), even though the tumor growth was similar to that of the irradiated-alone group. The combined treatment significantly reduced metastatic tumor masses in the lung and increased immune cell infiltration in primary tumor tissues. However, immune deficient nude mice with tumors did not exhibit prolonged survival in response to the combined treatment. Collectively, these results show that combined local irradiation and anti-neu antibody can elicit an immune-mediated abscopal effect to extend survival. Although the mechanism for abscopal effects induced by the combined treatment of radiation and anti-HER2/neu antibody was not elucidated, to our knowledge this is the first published study to describe the abscopal effect induced by the combination of local irradiation and the anti-HER2/neu antibody.

While the patent teaches enhancement of immunotherapy using immunization of endothelial cells modified in a manner to stimulate immunity to tumor endothelium, other means of abscopal effect stimulation involve non-immunological mechanisms. Mechanisms described below are discussed. In one study researchers examined whether abscopal effect was mediated through p53, a protein complex up-regulated in irradiated cells. Non-tumor-bearing legs of C57BL/6 (wild-type p53) and p53 null B6.129S2-Trp53(tm1Tyj) mice were irradiated to determine whether an abscopal effect could be observed against Lewis lung carcinoma (LLC) and T241 (fibrosarcoma) implanted at a distant site. In mice with wild-type p53, both LLC and T241 tumors implanted into the midline dorsum grew at a significantly slower rate when the leg of the animal was exposed to five 10-Gy fractions of radiation compared with sham-irradiated animals, suggesting that the abscopal effect is not tumor specific. When the radiation dose to the leg was reduced (twelve fractions of 2 Gy each), the inhibition of LLC tumor growth was decreased indicating a radiation-dose dependency for the abscopal effect. In contrast, when the legs of p53 null animals or wild-type p53 mice treated with pifithrin-alpha (a p53 blocker) were irradiated (five 10-Gy fractions), tumor growth was not delayed. These data implicate p53 as a key mediator of the radiation-induced abscopal effect and suggest that pathways downstream of p53 are important in eliciting this response.

Clinical use of combination checkpoint inhibitors for augmenting the abscopal effect have been described and incorporated by reference.

In some embodiments of the invention modification of neutrophil numbers and/or activity is performed in order to augment abscopal effect. This is further modified by placental vaccination. It has been observed that RT induced sterile inflammation with a rapid and transient infiltration of CD11b⁺Gr-1^{high+} neutrophils into the tumors. RT-recruited tumor-associated neutrophils (RT-Ns) exhibited an increased production of reactive oxygen species and induced apoptosis of tumor cells. Tumor infiltration of RT-Ns resulted in sterile inflammation and, eventually, the activation of tumor-specific cytotoxic T cells, their recruitment into the tumor site, and tumor regression. Finally, the concurrent administration of granulocyte colony-stimulating factor (G-CSF) enhanced RT-mediated antitumor activity by activating RT-Ns [40]. Augmentation of neutrophil activity may be performed by addition of various agents such as N-acetylcysteine intravenously at 0.2-200 micrograms per kilogram of body weight daily.

Intravenous vitamin C has been believed to induce anticancer responses through induction of oxidative stress in tumor cells. Combination of radiation and ascorbic acid has demonstrated augmentation of cancer cell death.

Cryosurgery has been demonstrated to stimulate immune responses as far back as 1969 in which a study was published demonstrating generation of agglutinating antibodies in patients with prostate cancer that underwent this procedure. Similar results where obtain in 1974 in an animal model of cancer treatment by cryosurgery. The ability of a localized induction of tumor cell death to induce a systemic antibody response was observed in a subsequent animal study in which an abscopal type systemic inhibition of tumor growth was observed subsequent to cryosurgery. The systemic effect induced by cryosurgery was similar to the abscopal effect that is observed in cancer when localized radiation is used to induce tumor cell death. Effects of radiation induced immunity may be amplified by macrophage activation.

In some embodiments of the invention dendritic cells are administered to the area of cryosurgery. Various means of generating dendritic cells are known in the art and are useful for the practice of the invention. For example, placental antigens may be used to pulse dendritic cells.

In one embodiment the invention teaches that administration of ValloVax, a placental endothelium-based vaccine described in the following references, is capable of sensitizing tumors to treatment with cancer vaccines, the cancer vaccines comprising of either peptide vaccines, protein vaccines, cellular vaccines, or endogenous vaccines. Without being bound to theory, the cancer endothelial targeting vaccines are capable of specifically inducing inactivation of tumor endothelial mediated lymphocyte death, thus allowing for cancer killing T cells to specifically enter the tumor and mediate tumor cell death. As substitution for ValloVax, other types of endothelial progenitor cells (EPC) may be used to stimulate immunity to tumor endothelium. The EPC, in one embodiment are a population of cells comprising cells having the surface marker CD44, cells having the surface marker CD13, cells having the surface marker CD90, cells having the surface marker CD105, cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s. The EPC may be treated in a manner to mimic the tumor microenvironment, specifically, they may be grown under the acidic conditions in the tumor microenvironment and are incorporated by reference. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation, and glutamine deprivation.

1. A method of increasing systemic antitumor effects of the immune system in a patient suffering from cancer comprising:
inducing tumor cell death in an immunogenic manner; and
administering at least one capable of stimulating an immune response which inhibiting/destroying tumor endothelial cells.

2. The method of claim 1, wherein the at least one agent capable of stimulating an immune response that inhibits/destroys tumor endothelial cells prior to induction of immunogenic cell death.

3. The method of claim 1, wherein the immunogenic cell death is necrosis.

4. The method of claim 1, wherein the immunogenic cell death is associated with release of mitochondria into the extracellular space.

5. The method of claim 1, wherein the immunogenic cell death is caused by at least one of cryosurgery, hyperthermia, localized irradiation, and chemotherapy.

6. The method of claim 1, wherein immunogenicity of cell death is augmented by pretreatment of the tumor with agents capable of augmenting markers of immunogenicity.

7. The method of claim 6, wherein the agents capable of augmenting markers of immunogenicity are selected from a group consisting of histone deacetylase inhibitors, DNA methyltransferase inhibitors, interferon alpha, interferon gamma, and lipoic acid.

8. The method of claim 1, wherein immunogenicity of cell death is accomplished by administration of dendritic cells prior to induction of immunogenic tumor cell death.

9. The method of claim 1, wherein the agent capable of inducing immune response towards tumor endothelial cells is an endothelial cell vaccine, wherein the endothelial cells are generated in a manner to resemble tumor endothelium.

10. The method of claim 9, wherein the agent capable of inducing immune response towards tumor endothelial cells possesses an antigenic moiety resembling proteins selected from a group consisting of ROBO 1-8, EGF-R, TEM-3, and CD105.

11. The method of claim 9, wherein the agent capable of inducing immune response towards tumor endothelial cells is derived from placental endothelial cells.

12. The method of claim 11, wherein the endothelial cells are cultured under at least one hypoxia and acidic conditions.

13. The method of claim 11, wherein the endothelial cells are cultured with interferon gamma to augment expression of HLA antigens.

14. The method of claim 11, wherein the endothelial cells are endothelial progenitor cells.

15. The method of claim 11, wherein the endothelial cells are allogeneic to the recipient.

16. The method of claim 11, wherein the endothelial cells are xenogeneic to the recipient.

17. The method of claim 1, wherein induction of immunity towards tumor endothelium is accomplished by immunization with ValloVax.

18. The method of claim 1, wherein induction of antibody responses is provoked towards tumor endothelium prior to induction of immunogenic cell death in order to enhance immunogenicity of the immunogenic cell death.

19. The method of claim 1, wherein immunity to tumor antigens is induced prior to induction of immunogenic cancer cell death.

20. The method of claim 19, wherein the immunity is induced by vaccination with immunization means selected from whole cell vaccines, peptide vaccine, c) mRNA vaccine, dendritic cell vaccine, and protein vaccine.
